(19) United States
(12) Patent Application Publication
(10) Pub. No.: US 2026/0334497 A1
(43) Pub. Date: Oct. 29, 2026

(54) FOODSTUFF

(71) Applicant: MARLOW FOODS LIMITED, Stokesley (GB)

(72) Inventors: Albertus Antonius Gerardus HAFKAMP, Stokesley (GB); Linda Christina PRAVINATA, Stokesley (GB); Willem Cornelis MEIJER, Stokesley (GB); Anna Jayne TAYLOR, Stokesley (GB)

(21) Appl. No.: 19/487,344

(22) PCT Filed: May 7, 2024
(86) PCT No.: PCT/GB2024/051184

§ 371 Date: Nov. 24, 2025

(30) Foreign Application Priority Data
May 25, 2023 (GB) 2307836.3

(51) Int. Cl. A23C 20/02; A23L 31/00

(52) U.S. Cl. CPC A23C 20/02; A23L 31/00

(57) ABSTRACT

A foodstuff, for example a soft, vegan cheese, comprises a filamentous fungus, for example Fusarium venenatum and a vegetable oil with a melting point of at least 20 °C, wherein the foodstuff has a firmness of less than 10000 g (preferably less than 4000 g).

This invention relates to a foodstuff and particularly, although not exclusively, relates to a cheese-like foodstuff which includes no animal products and is therefore suitable for vegans. Especially preferred embodiments relate to a cheese-like foodstuff which is similar in a range of properties to soft milk-based cheese.

Soft cheeses (i.e. animal milk-based traditional cheeses), such as Danish blue, stilton, Roquefort, camembert and brie are well known. They have a range of desirable properties such as soft texture, good meltability, mild cheese and/or a slight mouldy taste and they are visually appealing.

It is desirable to produce soft, cheese-like, vegan foodstuffs. However, this is challenging. It is known to produce cheese-like foodstuffs for vegans incorporating a range of different proteins including, for example, soy protein. However, it is difficult to produce cheese-like vegan foodstuffs which have high levels of protein and characteristics which largely correspond to those of traditional soft cheeses. For example, it is very challenging to produce a cheese-like vegan foodstuff which closely replicates a soft cheese in having appropriate softness, meltability, colour (when raw or cooked) and a natural umami flavour.

It is an object of preferred embodiments of the present invention to address the above-mentioned problems.

It is an object of preferred embodiments of the present invention, to provide a soft cheese-like foodstuff which is suitable for vegans.

It is an object of preferred embodiments of the present invention to provide a cheese-like foodstuff which is visually attractive and desirable in the raw state or when cooked.

According to a first aspect of the invention, there is provided a foodstuff, for example a vegan cheese, the foodstuff comprising:
a fungus; and
an oil.

Said oil is suitably primarily composed of fatty acids in the form of esters. References below to an oil which is composed of or includes a fatty acid includes the fatty acid as an ester (e.g. the oil include a fatty acid residue), rather than the referenced fatty acid being a free fatty acid in the oil.

Said oil is preferably a vegetable oil.

Said oil may have a melting point of at least 20 °C, preferably at least 22 °C. The melting point may be less than 40 °C, preferably less than 30 °C, more preferably less than 27 °C.

Said oil has preferably not been chemically treated to functionalise it. It is preferably not a hydrogenated version of a natural oil.

The % of components in an oil as described herein may be assessed by GC-HRMS or alternative techniques known to a skilled person in the art.

Said oil may include at least 60 wt %, preferably at least 70 wt %, more preferably at least 78 wt %, of saturated fatty acids. Said oil may include less than 95 wt %, preferably less than 90 wt %, of saturated fatty acids.

Said oil may include at least 4 wt % of monounsaturated fatty acids. Said oil may include less than 10 wt %, preferably less than 8 wt %, of monounsaturated fatty acids.

Said oil may include less than 5 wt %, preferably less than 3 wt %, of polyunsaturated fatty acids.

Said oil may include at least 40 wt %, preferably at least 45 wt %, of lauric acid. Said oil may include less than 65 wt %, preferably less than 60 wt %, of lauric acid.

Said oil may include at least 15 wt % of myristic acid. Said oil may include less than 30 wt %, preferably less than 25 wt %, of myristic acid.

Said oil may include at least 6 wt % of palmitic acid. Said oil may include less than 15 wt %, preferably less than 11 wt %, of palmitic acid.

Said oil may include at least 4 wt % of oleic acid. Said oil may include less than 10 wt %, preferably less than 8 wt %, of oleic acid.

Said oil may include at least 2 wt %, preferably at least 3 wt, of stearic acid. Said oil may include less than 5 wt %, of stearic acid.

Said oil may include at least 4 wt % of capric acid. Said oil may include less than 10 wt %, preferably less than 8 wt %, of capric acid.

Said oil may include at least 1 wt %, preferably at least 2 wt %, of linoleic acid. Said oil may include less than 5 wt %, of linoleic acid.

Any grade of said oil, for example virgin, extra virgin, highly refined or winterized, may be selected.

Said oil is preferably coconut oil.

Said oil preferably makes up at least 80 wt %, preferably at least 90 wt %, more preferably at least 95 wt % or, especially, at least 99 wt % of the total wt % of all oils in said foodstuff, excluding any oil or fat in the fungus. Said oil may be the only oil in the foodstuff, excluding any oil or fat in the fungus.

Said foodstuff may include at least 15 wt %, suitably at least 21 wt %, preferably at least 24 wt %, more preferably at least 26 wt %, of said oil. Said foodstuff may include less than 40 wt %, preferably less than 35 wt %, more preferably less than 32 wt % of said oil.

The sum of the wt % of all oils and fats in said foodstuff excluding any oil/fat in the fungus may be at least 21 wt %, preferably at least 24 wt %, more preferably at least 26 wt %. The sum of the wt % of all oils and fats in said foodstuff excluding any oil/fat in the fungus may be less than 40 wt %, preferably less than 35 wt %, more preferably less than 32 wt %.

Unless otherwise stated herein, the amount of fungus is on a dry mass basis. That is the weight on a dry mass basis excludes any water which may be incorporated into a mass comping the fungus.

Said fungus may include at least 35 wt %, preferably at least 40 wt %, protein on a dry mass basis.

Said fungus may include less than 65 wt %, preferably less than 60 wt %, protein on a dry mass basis.

Said fungus may include at least 10 wt %, preferably at least 15 wt %, dietary fibre on a dry mass basis. Said fungus may include less than 30 wt %, preferably less than 23 wt %, dietary fibre on a dry mass basis.

Said fungus may include at least 4 wt %, preferably at least 7 wt %, fat on a dry mass basis. Said fungus may include less than 20 wt %, preferably less than 10 wt %, fat on a dry mass basis.

Preferably, said fungus is a filamentous fungus.

Said fungus may comprise filaments. Said filaments may have lengths of less than 500 μm, preferably less than 100 μm. Said filaments may have a length greater than 1 μm, for example greater than 10 μm. Preferably, fewer than 5 wt %, preferably substantially no, filaments of said filamentous fungus have lengths of greater than 500 μm; and preferably fewer than 5 wt %, preferably substantially no filaments have lengths of greater than 250 μm. Preferably, values for the number average of the lengths of said filaments are also as stated above. Thus, the number average of the lengths of said filaments may be in the range 1 μm to 100 μm, preferably in the range 2 μm to 80 μm.

Said filamentous fungus may comprise filaments having diameters of less than 20 μm, preferably less than 10 μm, more preferably 5 μm or less. Said filaments may have diameters greater than 1 μm, preferably greater than 2 μm. Preferably, values for the number average of said diameters of said filaments are also as stated above. Thus, the number average of said diameters of said filaments are preferably in the range 1 μm to 20 μm.

Said filamentous fungus may comprise filaments having an aspect ratio (length/diameter) of less than 500, preferably less than 200, more preferably less than 100. The aspect ratio may be greater than 5, preferably greater than 10. Preferably, values for the average aspect ratio of said filaments (i.e. the number average of the lengths of the filaments divided by the number average of the diameters of the said filaments) are also as stated above. Thus, the number average of the lengths of the filaments divided by the number average of the diameters of the said filaments is preferably in the range 5 to 500, more preferably in the range 10 to 100.

Preferably, said filamentous fungus comprises fungal mycelia and suitably at least 80 wt %, preferably at least 90 wt %, more preferably at least 95 wt % and, especially, at least 99 wt % of said fungus comprises fungal mycelia. Some filamentous fungi may include both fungal mycelia and fruiting bodies. Said filamentous fungus preferably comprise a filamentous fungus of a type which does not significant produce fruiting bodies.

Said filamentous fungus preferably comprises fungus selected from fungi imperfecti.

Preferably, said filamentous fungus comprises, and preferably consists essentially of, cells of Fusarium species, especially of Fusarium venenatum A3/5 (formerly classified as Fusarium graminearum) (IMI 145425; ATCC PTA-2684 deposited with the American Type Culture Collection, 10801 University Boulevard, Manassas, VA.).

Preferably, said filamentous fungus is non-viable. Preferably, said filamentous fungus has been treated to lower the level of RNA which it contains. Thus, the level of RNA in said filamentous fungus is preferably less than the level in an identical fungus when in a viable state. The level of RNA in the filamentous fungus is preferably less than 2 wt % on a dry mass basis.

Said foodstuff suitably includes at least 1.0 wt %, preferably at least 2.0 wt %, of said fungus on a dry mass basis. Said foodstuff may include less than 20.0 wt %, suitably less than 15.0 wt %, preferably less than 10.0 wt %, more preferably less than 8.0 wt %, of said fungus on a dry mass basis.

Said foodstuff suitably includes at least 1.0 wt %, preferably at least 2.0 wt %, of protein on a dry mass basis. Said foodstuff may include less than 20.0 wt %, suitably less than 10.0 wt %, preferably less than 9.0 wt %, more preferably less than 7.0 wt %, of protein on a dry mass basis. The protein level may be assessed by the Kjeldahl method.

Preferably, in said foodstuff, the sum of the wt % of said fungus on a dry mass basis and said oil is at least 25 wt %, preferably at least 30 wt %, more preferably at least 32 wt %. Said sum may be less than 40 wt %, preferably less than 37 wt %.

Preferably, in said foodstuff, the sum of the wt % of each fungus on a dry mass basis and each oil/fat is at least 25 wt %, preferably at least 30 wt %, more preferably at least 32 wt %. Said sum may be less than 40 wt %, preferably less than 37 wt %.

A reference herein to the weight of water in said composition suitably refers to the total water content in the composition irrespective of its origin.

Said foodstuff may include at least 35 wt %, preferably at least 40 wt %, more preferably at least 45 wt %, especially at least 49 wt % of water. The amount of water in said foodstuff may be less than 60 wt %.

In said foodstuff, suitably, a ratio defined as the weight of said oil divided by the weight of water is greater than 0.2, preferably is greater than 0.4. The ratio may be less than 3.0, preferably less than 2.5, more preferably less than 2.0.

In said foodstuff, suitably, a ratio defined as the total weight of oil(s)/fats(s) divided by the weight of water is greater than 0.2, preferably is greater than 0.4. The ratio may be less than 2.0, preferably less than 1.0, more preferably less than 0.8.

In said foodstuff, the sum of the wt % of said fungus, oil and water may be at least 60 wt %, preferably at least 75 wt %, more preferably at least 84 wt %. Said sum may be less than 95 wt %, preferably less than 90 wt %.

In said foodstuff, the sum of the wt % of each fungus, each oil/fat and water may be at least 60 wt %, preferably at least 75 wt %, more preferably at least 84 wt %. Said sum may be less than 95 wt %, preferably less than 90 wt %.

Said foodstuff may include: 2 to 15 wt %, preferably 3 to 6 wt %, of said fungus on a dry mass basis; and 20 to 40 wt %, preferably 25 to 35 wt %, of an oil.

The balance may include other ingredients including water and optionally one or more starches.

Said foodstuff suitably includes one or more starches. The sum of the wt % of starches in said foodstuff may be at least 4.0 wt %, preferably at least 8.0 wt %. Said sum may be less than 20.0 wt %, preferably less than 14.0 wt %.

The level of amylose in the one or more starches may affect the softness of a foodstuff described.

For example, starches which have higher levels of amylose (eg greater than 10.0 wt %, greater than 15.0 wt % or greater than 12.0 wt %) may result in production of harder foodstuffs. Starches which include lower levels of amylose (eg less than 10.0 wt %) (eg waxy corn, rice or tapioca starches) may be preferred.

To produce a soft cheese, said foodstuff may include at least 4.0 wt % or at least 7.0 wt % (and suitably less than 15.0 wt % or less than 10.0 wt %) of a starch which has a relatively low level (eg less than 10.0 wt %, less than 5.0 wt % or less than 1.0 wt %) of amylose.

In one preferred embodiment, said foodstuff may include a starch which is relatively high in amylopectin. Said foodstuff may include a starch which has at least 10 wt % or at least 20% wt % amylopectin. Such a starch may include less than 10 wt % or less than 5 wt % amylose.

Said starch is preferably vegetable-derived.

Said starch is preferably a rice starch, for example a waxy ice starch.

In said foodstuff, the sum of the wt % of each fungus, each oil/fat, each starch and water may be at least 70.0 wt %, preferably at least 80.0 wt %, more preferably at least 90.0 wt %, especially at least 93.0 wt %. Said sum may be less than 100 wt %, preferably less than 99.0 wt %, more preferably less than 98.0 wt %.

Said foodstuff may include one or more flavourants and/or colourants. For example, the sum of the wt % of flavourants and colourants in the foodstuff may be at least 1.0 wt %, preferably at least 2.0 wt %. Said sum may be less than 6.0 wt %.

Said foodstuff may include 0 to 5 wt % of edible hydrocolloid. Suitably, however, said foodstuff includes less than 4.0 wt %, preferably less than 2.0 wt %, more preferably less than 1.0 wt % and, especially, less than 0.5 wt % of hydrocolloids in total. It may include less than 0.1 wt % or even about 0 wt % of hydrocolloids in total.

Said foodstuff may include less than 4.0 wt %, preferably less than 2.0 wt %, more preferably less than 1.0 wt % and, especially, less than 0.5 wt % of non-starch based carbohydrate polymers in total. It may even include less than 0.1 wt % or even about 0 wt % of non-starch based carbohydrate polymers in total.

Said foodstuff may include less than 4.0 wt %, preferably less than 2.0 wt %, more preferably less than 1.0 wt % and, especially, less than 0.5 wt % of sucrose. It may include less than 0.1 wt % or even about 0 wt % of sucrose.

In some cases, an acid, for example a food grade acid, such as lactic acid or propionic acid (lactic acid being preferred) may be added to the foodstuff to reduce the pH of the foodstuff to less than 4.5, Said acid may increase shelf-life of the foodstuff and/or also act to soften the texture of the foodstuff. In a preferred embodiment, lactic acid is added to reduce the pH of the foodstuff.

Said foodstuff may include less than 4.0 wt %, preferably less than 2.0 wt %, more preferably less than 1.0 wt % and, especially, less than 0.5 wt % of lactic acid. It may include less than 0.1 wt % or even about 0 wt % of lactic acid.

Said foodstuff may include less than 4.0 wt %, preferably less than 2.0 wt %, more preferably less than 1.0 wt % and, especially, less than 0.5 wt % of lactic acid producing bacteria. It may include less than 0.1 wt % or even about 0 wt % of lactic acid producing bacteria.

Said foodstuff may include less than 4.0 wt %, preferably less than 2.0 wt %, more preferably less than 1.0 wt % and, especially, less than 0.5 wt % of bacteria. It may include less than 0.1 wt %, less than 0.01 wt % or even about 0 wt % of bacteria.

Said foodstuff may include one or more mineral additives (in addition to any mineral additive which includes a Group I metal), for example, calcium, magnesium or ferrous salts. Said foodstuff preferably includes less than 2.0 wt %, preferably less than 1.0 wt %, more preferably less than 0.1 wt % and, especially, less than 0.05 wt % of said one or more mineral additives. It may include less than 0.01 wt % or even about 0 wt % of said one or more mineral additives.

Said foodstuff suitably has a protein content of at least 2 g/100 g, preferably at least 3 g/100 g of protein. The protein content may be less than 1 g/100 g or less than 8 g/100 g.

Said foodstuff preferably includes 0 wt % of ingredients of animal origin. Said foodstuff is preferably suitable for vegans. Said foodstuff is preferably a cheese-substitute which includes no animal products.

In said foodstuff, the sum of the wt % of each fungus, each oil/fat, each starch, each flavourant, salt (eg NaCl) and water may be at least 80.0 wt %, preferably at least 85.0 wt %, more preferably at least 90.0 wt %, especially at least 95 wt %. Said sum may be 100 wt % or less.

Said foodstuff may be in the form of a block. The block may have a volume of at least 25 cm³, preferably at least 100 cm³. The volume may be less than 1000 cm³ or less than 500 cm³. The block may have at least one opposing pair of substantially planar faces.

Said foodstuff, for example said block, may have a firmness, assessed as described in Test 1, of less than 10000 g, less than 8000 g, less than 6000 g, or less than 4000 g. The firmness may be at least 1000 g or at least 1500 g or at least 2000 g. Firmness is suitably assessed at 20 °C.

Said foodstuff is preferably provided in a package. For example, a said block may be provided in said package. The package may be arranged to restrict passage of oxygen to the foodstuff therein. Said package may comprise an oxygen barrier material. Said package may comprise a plastics packaging material. Said foodstuff is preferably the only foodstuff contained in the package.

According to a second aspect of the invention, there is provided a process for making a foodstuff, the process comprising mixing a fungus and an oil.

Said foodstuff may have any feature of the foodstuff of the first aspect. Said fungus may have any feature of the fungus of the first aspect. Said oil may have any feature of the oil of the first aspect.

In a step (i), a biomass comprising said fungus is suitably selected. Said biomass may be in the form of a paste. Said biomass may include 20-30 wt %, (preferably 22-26 wt %) of fungus on a dry mass basis and 70-80 wt %, (preferably 74 to 78 wt %) of water.

Said process may include contacting the fungus with said oil, suitably with mixing. Said process may comprise making an emulsion comprising water, fungus and said oil.

Said process may include contacting the fungus with a starch as described in the first aspect. Preferably, said emulsion comprising water, fungus and said oil is contacted with said starch.

Said process may include contacting the fungus with one or more flavourants and/or colourants as described according to the first aspect.

Said process may comprise introducing a mixture prepare in the process into a mould, for example to form said mixture into a predetermined shape, for example into a block.

Said process is preferably for producing a foodstuff having a firmness, suitably assessed as described in Test 1, as described in the first aspect. The firmness may be less than 10000 g, less than 4000 g or less than 3000 g.

Said process is preferably for producing a foodstuff which is a soft vegan cheese.

Any feature of any aspect of any invention described herein may be combined with any feature of any other invention described herein mutatis mutandis.

Specific embodiments of the invention will now be described, by way of example, with reference to the accompanying figures in which:

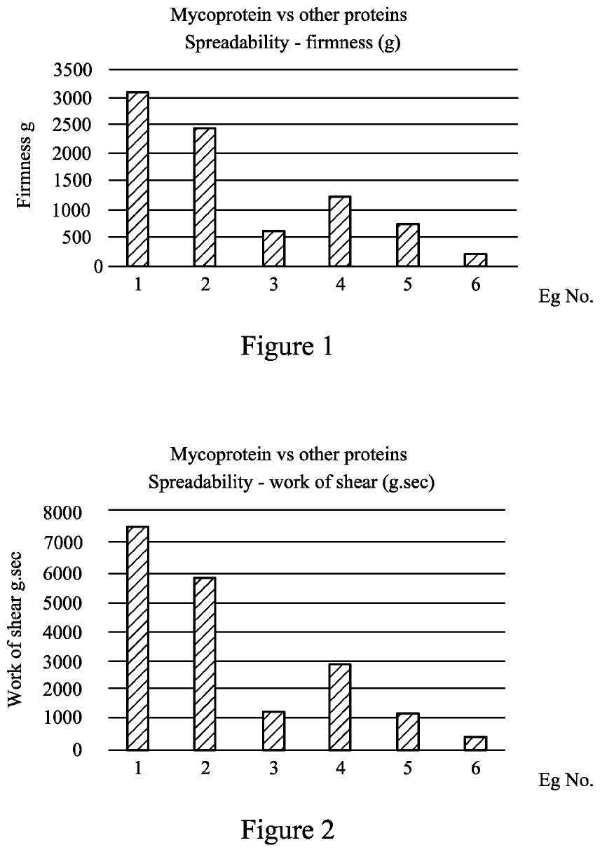

FIG. 1 is a graph comparing spreadability (firmness(g)) of the cheeses of Examples 1 to 6;

FIG. 2 is a graph comparing spreadability (work of shear (g·sec)) of the cheeses of Examples 1 to 6;

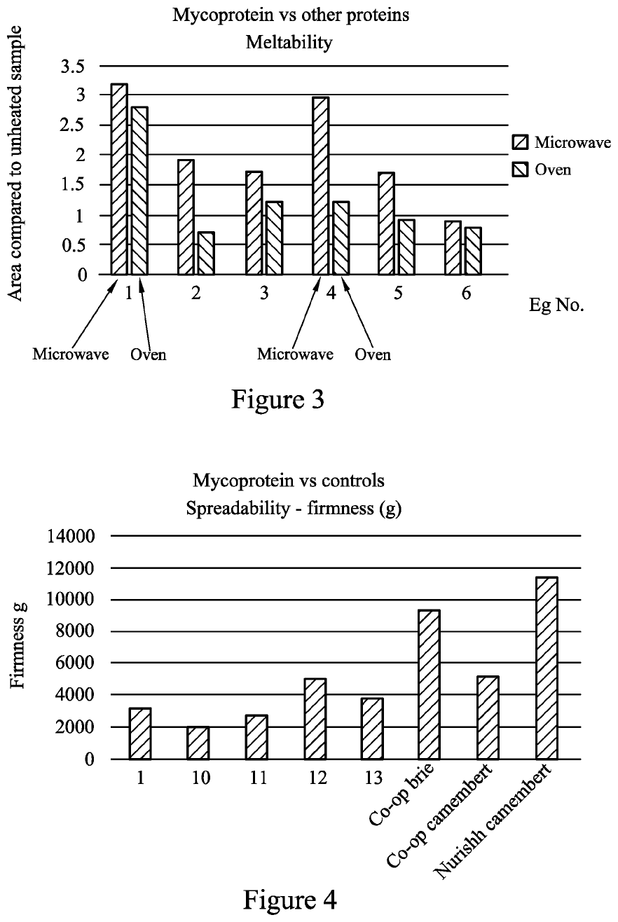

FIG. 3 is a graph comparing meltability of the cheeses of Examples 1 to 6;

FIG. 4 is a graph comparing spreadability (firmness(g)) of the cheeses of Examples 1, 10 to 13 and commercially available cheeses;

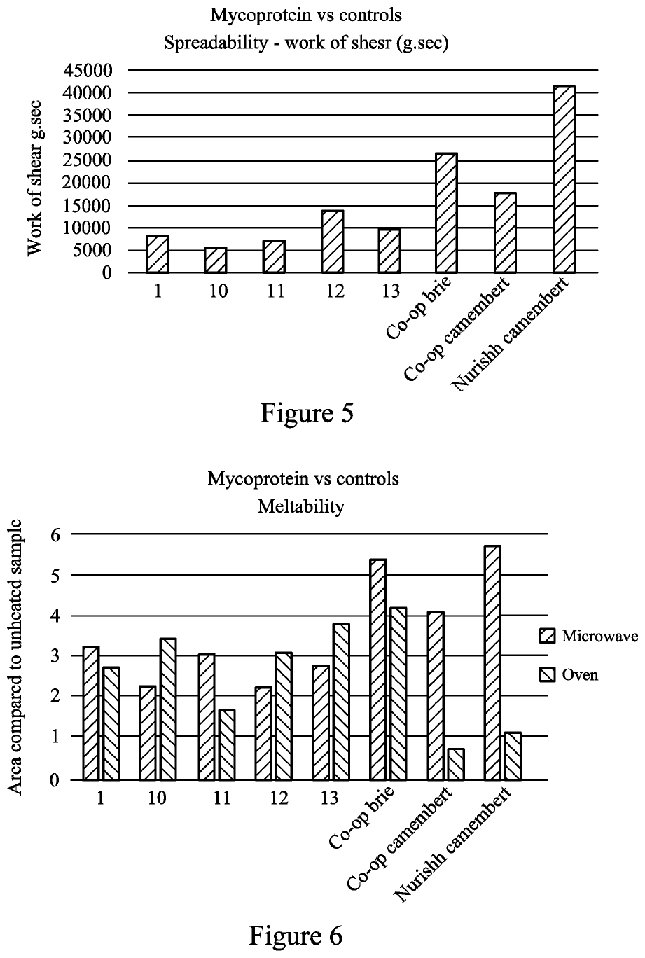

FIG. 5 is a graph comparing spreadability (work of shear (g·sec)) of the cheeses of Examples 1, 10 to 13 and commercially available cheeses; and

FIG. 6 is a graph comparing meltability of the cheeses of Examples 1, 10 to 13 and commercially available cheeses.

The following materials are referred to herein:

Mycoprotein paste—Mycoprotein paste—refers to a visco-elastic material comprising a mass of edible filamentous fungus derived from Fusarium venenatum A3/5 (formerly classified as Fusarium graminearum Schwabe) (IMI 145425; ATCC PTA-2684 deposited with the American type Culture Collection, 12301 Parklawn Drive, Rockville Md. 20852) and treated to reduce its RNA content to less than 2% by weight by heat treatment. Further details on the material are provided in WO96/21362 and WO95/23843. The material may be obtained from Marlow Foods Limited of Stokesley, U.K. It comprises about 23-25 wt % solids (the balance being water) made up of non-viable RNA reduced fungal hyphae of approximately 400-750 μm length, 3-5 μm in diameter and a branching frequency of 2-3 tips per hyphal length.

Coconut oil—Kirkland™ Coconut Oil Unrefined Virgin Cold Pressed 100% Pure, having a melting point of about 24 °C. An alternative is CocoPacific™ Odourless Raw Extra Virgin Coconut Oil.

Corn starch—a corn starch with an amylose content of about 25 wt %.

Rice starch—a waxy rice starch with a negligible amylose content.

Potato protein—a potato protein with a total protein content of 90.5 g/100 g.

Nutritional yeast—a commercially-available yeast.

Pea protein—Pisane™ M9, from Fibrisol™, having a protein content of 81.7 g/100 g.

Faba protein—product from Australian Plant Protein, having a protein content of 82.1 g/100 g.

Soy protein—product having a protein content of 90.0 g/100 g.

The following tests and assessments are referred to herein.

### Test 1—Texture Analysis—Spreadability

A TTC Spreadability Rig from Stable Microsystems was used to assess the spreadability (which may also be considered to define firmness) of foodstuffs prepared as described herein. The rig measures the ease with which a product can be applied in a thin, even layer. It comprises a male 90 degrees cone probe and five precisely matched female acrylic cone shaped product holders.

The material is either deposited and allowed to set up in the lower cone holders in advance of testing or is filled with a spatula and then the surface levelled. The product is forced to flow outward at 45 degrees between the male and female cone surfaces during the test, the ease of which indicates the degree of spreadability. The test was undertaken at ambient temperature (eg 20 °C+/−5 °C).

The settings for the rig were as follows:

Test 2 - Sensory Analysis

| Caption | Value | Units |
|---|---|---|
| Test Mode | Compression | |
| Pre-Test Speed | 8.00 | mm/sec |
| Test Speed | 1.00 | mm/sec |
| Post-test speed | 5.00 | mm/sec |
| Target Mode | Distance | |
| Distance | 17.000 | mm |
| Trigger Type | Auto (Force) | |
| Trigger Force | 0.5 | g |

| | |
|---|---|
| Break Mode | Off |
| Stop Plot At | Start Position |
| Tare Mode | Auto |
| Advanced Options | On |

A panel was assembled in accordance with_ISO 8586:2012(E) (Corrected version 2014 Jun. 15) and trained to undertake a range of sensory tests on selected foodstuffs. In particular, a blind tasting of samples was undertaken.

### Test 3—Colour on Cooking

Slices were heated in a frying pan on medium heat to test for colour on cooking and in particular browning.

### Test 4—Assessment of Meltability of Cheeses

In general terms, meltability of samples was assessed by comparing the area of a sample before and after heating. In the method, samples were cut from a mass into discs of 25 mm diameter and 11 mm height. The discs were placed within suitable trays and heated either in a conventional thermal oven at 180 °C for five minutes or in a microwave oven at 800 W for 10 seconds if an individual disc was assessed; or at 800 W for 20 seconds if four discs were assessed together. Images of the discs before and after heating were captured using a Samsung S10 plus camera and images exported to commercially available Image J™ software. The meltability was based on the expansion of the discs, calculated by:

$$\vert \frac{{A}_{unheated} - {A}_{heated}}{{A}_{unheated}} \vert$$

where A is the area.

### Test 5—Assessment of Total Water Content of Cheeses

The moisture % of cheeses was assessed by weighing 3 g of a sample and then re-weighing during removal of moisture by heating to 130 °C until no more weight was lost. The difference between the initial weight and final weight was used to calculate moisture content.

### Example 1—Preparation of Soft, Vegan Cheese

A vegan cheese was made from the ingredients in the table below.

| Ingredient | wt % |
|---|---|
| Mycoprotein paste | 27 |
| Water | 33.92 |
| Coconut oil | 27.08 |
| Salt | 2 |
| Rice starch | 10 |

The example used a microfluidised mycoprotein paste. The preparation of the microfluidised paste involved homogenising chilled, fresh mycoprotein paste with cold tap water in a Thermomix™ mixer (ratio of 1 part paste to 2 parts water). This mixture was then sent through a Microfluidics™ microfluidizer at approximately 900 bar. The resultant milk (comprising 7.77 wt % solids) was then homogenised in the Thermomix mixer again, and additional paste was added to adjust the ratio to 1 part paste to 1 part water, the mixture then comprising 11.14 wt % solids.

Next in the preparation, the microfluidised paste and coconut oil were mixed at high speed in a Thermomix (Trade Mark) mixer at 37 °C for a minimum of 5 minutes until a homogenous, smooth mix was formed. Dry ingredients were then added to the mixer and mixing continued at low speed for a further minute. Then the mixture was heated to 85 °C at mid to high speed to gelatinize the starch and yield a smooth, homogeneous mix. It could take between 5 and 10 minutes for the sample to reach temperature. The mixture was then poured into a mould in the form of a foil or silicone tray and chilled in a standard fridge at a temperature of 5 °C.

### Examples 2 to 6—Preparation of Alternate Cheeses Using Other Common Protein-Containing Ingredients or No Protein-Containing Ingredient (Comparative Examples)

Cheeses were made using other non-animal based protein-containing ingredients (or no protein-containing ingredients) and compared to the vegan cheese of Example 1.

Cheeses were prepared having the ingredients in the table below.

Example No.
wt %

| Ingredient | 2 | 3 | 4 | 5 | 6 |
|---|---|---|---|---|---|
| Water | 56.92 | 56.52 | 56.52 | 56.92 | 60.92 |
| Coconut oil | 27.08 | 27.08 | 27.08 | 27.08 | 27.08 |
| Salt | 2 | 2 | 2 | 2 | 2 |
| Rice starch | 10 | 10 | 10 | 10 | 10 |
| Potato | 4 | 0 | 0 | 0 | 0 |
| protein | | | | | |
| Pea protein | 0 | 4.4 | 0 | 0 | 0 |
| Faba protein | 0 | 0 | 4.4 | 0 | 0 |
| Soy protein | 0 | 0 | 0 | 4 | 0 |

In the preparation, the water and coconut oil were mixed at high speed in a Thermomix (Trade Mark) mixer at 37 °C for a minimum of 5 minutes until a homogenous, smooth mix was formed. Then, the selected protein, starch and salt were added to the mixer and mixing continued at low speed for a further minute. Then the mixture was heated to 85 °C at mid to high speed to gelatinize the starch and yield a smooth, homogeneous mix. It could take between 5 and 10 minutes for the sample to reach temperature. The mixture was then poured into a mould in the form of a foil tray and chilled in a standard fridge at a temperature of 5 °C.

The table below includes information on the levels of specified components and characteristics of the vegan cheeses of Examples 1 to 6. Nutritional information was calculated from the raw ingredients' nutrient data having regard to the wt % included in the cheeses.

| | Example No. | | | | | |
|---|---|---|---|---|---|---|
| Characteristic | 1 | 2 | 3 | 4 | 5 | 6 |
| Energy (kcal) | 299.8 | 292.4 | 295.7 | 293.1 | 277.5 | 250.3 |
| Protein g/100 g | 3.6 | 3.7 | 3.6 | 3.7 | 3.7 | 0.1 |
| Fat -Total | 27.5 | 27.1 | 27.5 | 27.4 | 27.2 | 27.1 |
| g/100 g | | | | | | |
| Carbohydrate - | 9.2 | 8.8 | 8.8 | 9.0 | 8.8 | 8.8 |
| Total g/100 g | | | | | | |
| Fibre -Total | 1.6 | 0 | 0.1 | 0 | 0.1 | 0 |
| g/100 g | | | | | | |
| Salt | 2.0 | 2.1 | 2.2 | 2.1 | 2.1 | 2.0 |
| Solids | 48.09 | 42.77 | 43.70 | 44.01 | 44.31 | 25.55 |

### Example 7—Testing of Cheeses of Examples 1 to 6

A texture profile analysis of each cheese of Examples 1 to 6 was undertaken as described in Test 1 and results are provided in FIGS. 1 and 2 which show that the firmness of the vegan cheese of Example 1 approximates to that of soft dairy cheeses, whereas the vegan cheeses of examples 2 to 6 are overly soft.

### Example 8—Meltability of Cheeses of Examples 1 to 6

The cheeses of examples 1 to 6 were subjected to test 4 to assess meltability and results are provided in FIG. 3 which shows the cheese of example 1 has high meltability when using a thermal oven and a microwave oven in comparison to the cheeses of examples 2 to 6 which have inferior meltability.

### Example 9—Other Tests Undertaken on Cheeses of Examples 1 to 6

Tests 2 and 3 were undertaken on samples of the cheeses of Examples 1 to 6 and observations are summarised in the table below.

| | Example number | | | | | |
|---|---|---|---|---|---|---|
| Test | 1 | 2 | 3 | 4 | 5 | 6 |
| Visual | Beige/ | Brown, | Beige/cream | Greenish | Cream | Solid oil |
| | cream, | appears | | cream | | separated on |
| | smooth | grainy | | | | top, very soft |
| | | | | | | gel |
| | | | | | | underneath |
| Frying | Golden | Easily burnt, | Decent spread, | Decent | Good | Oil |
| | brown, | dark brown on | some bubbling, | spread, | spread, | immediately |
| | cheese | fried side, | appears dry, | quickly | neutral | melted away |
| | aroma, | fishy notes, | pea notes, | formed a | smell, good | from gel |
| | spreads | little spread, | okay browning | film on top | bubbling, | component, |
| | well, good | no bubbling, | on fried side | so the | and golden | gel spread |
| | bubbling | appears dry | | sample | brown on | quickly and |
| | (like | | | bubbled, | fried side | mostly |
| | cheese) | | | but air | | disappeared |
| | | | | could not | | |
| | | | | escape, | | |
| | | | | beany | | |
| | | | | notes, | | |
| | | | | anemic on | | |
| | | | | fried side, | | |
| | | | | top side | | |
| | | | | remained | | |
| | | | | very moist | | |
| Taste | Light | Bitty texture, | Smooth | Smooth | Smooth | Separated oil |
| (raw) | cheese or | strong off- | texture, but | texture, | texture, fairly | slightly |
| | savoury | notes | leaves a | strong | neutral taste | coconutty, |
| | notes, | | “starchiness” in | bean off- | with some | gel section |
| | smooth | | the mouth. | notes | off-notes | salty with no |
| | texture | | Strong pea off- | | | other notes. |
| | | | notes | | | |
| Taste | Cheesy | Texture | Dry texture, | Sample | Neutral | Bland |
| (fried) | flavour | similar to a | pea flavour | retained | flavour, | |
| | and chew | stale cracker, | | moisture, | decent | |
| | | strong fish off- | | bean off- | texture (crisp | |
| | | notes | | notes | underneath; | |
| | | | | | not too wet), | |
| | | | | | but a little | |
| | | | | | dry | |

### Examples 10 to 16—Preparation of Other Mycoprotein-Containing Soft Vegan Cheeses and Comparison with Commercially-Available Cheeses

Following the procedure generally described for Example 1, vegan cheeses of Examples 10 to 13 were prepared having the ingredients detailed in the table below.

Example No.
wt %

| Ingredient | 10 | 11 | 12 | 13 |
|---|---|---|---|---|
| Mycoprotein | 22 | 22 | 21 | 17.5 |
| paste | | | | |
| Water | 44 | 42 | 38 | 36.5 |
| Coconut oil | 22 | 25 | 25 | 30 |
| Salt | 2 | 2 | 1 | 1 |
| Rice starch | 10 | 10 | 20 | 10 |
| Nutritional yeast | 0 | 0 | 5 | 5 |

The table below includes information on the levels of specified components and characteristics of the vegan cheeses of Examples 10 to 13, with relevant characteristics being assessed as described in Tests 1 to 5 as applicable. In addition, the table details the levels of specified components and characteristics of commercially-available soft dairy cheeses (COOP™ Brie (Example 14) and COOP™ Camembert (Example 15) and a soft vegan cheese (NURISHH™ camembert (Example 16)).

| | Example No. | | | | | | |
|---|---|---|---|---|---|---|---|
| Characteristic | 10 | 11 | 12 | 13 | 14 | 15 | 16 |
| Energy (kcal) | 250.3 | 276.3 | 293.7 | 335.5 | 291.0 | 290.0 | 286.0 |
| Protein g/100 g | 2.9 | 2.8 | 5.1 | 4.7 | 18.0 | 22.0 | 1.0 |
| Fat -Total | 22.3 | 25.3 | 25.6 | 30.5 | 24.0 | 23.0 | 22.0 |
| g/100 g | | | | | | | |
| Carbohydrate - | 9.1 | 9.1 | 10.1 | 10.0 | 0.5 | 0 | 21.0 |
| Total g/100 g | | | | | | | |
| Fibre -Total | 1.3 | 1.2 | 2.2 | 2.0 | 0 | 0 | 0 |
| g/100 g | | | | | | | |
| Salt | 2.0 | 2.0 | 1.0 | 1.0 | 1.3 | 1.5 | 1.5 |
| Solids wt % | 39.66 | 42.7 | 48.42 | 49.46 | 47.40 | 46.84 | 44.42 |

A texture profile analysis of each cheese of Examples 10 to 17 was undertaken as described in Test 1 and results are provided in FIGS. 4 and 5.

In addition, meltability was assessed as described in test 4 and results are provided in FIG. 6.

### Example 17—Preparation of Soft, Vegan Cheese

Following the procedure generally described for Example 1, a vegan cheese was prepared having the ingredients detailed in the table below.

| Ingredient | wt % |
|---|---|
| Mycoprotein paste | 28.35 |
| Water | 36.15 |
| Coconut oil | 18.0 |
| Salt | 1.0 |
| Corn starch | 1.5 |
| Rice starch | 15.0 |

The cheese is found to be an acceptable soft cheese.

### Example 18—Preparation of Soft, White Mould, Vegan Cheese

Following the procedure generally described for Example 1, a vegan cheese was prepared. The following steps were undertaken:

1 After heating to 85 °C, as described in Example 1, and with the cheese at a temperature of about 50 °C before being poured into a mould, lactic acid (80% E270) was added to the cheese to adjust the pH to 4.50. In addition, the lactic acid acts as a carbon source for the mould which is subsequently added to create a white surface.

2 A culture of Penicillium candidum (0.05 units/liter) was added when the cheese curd was at approximately 40 °C and mixed thoroughly at high speed.

4 The cheese was filled into a mould or a casing and stored at 4-7 °C to harden.

5 After 2 weeks, the mould was removed, and the cheese was ripened in a ripening chamber at 13 °C under high moisture conditions.

6 After 7-10 days of ripening, the surface of the cheese was completely covered with white mould.

7 The mould covered cheese was packed and ready for consumption.

The cheese was found to be camembert-like.

In general terms, the results show that vegan cheeses as described herein containing mycoprotein paste can be matched with commercially available milk-based cheeses. The mycoprotein-based vegan cheeses advantageously deliver high levels of protein and have excellent taste, texture, softness and aesthetic appeal in raw and cooked states.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A foodstuff, for example a vegan cheese, the foodstuff comprising:
a fungus; and
an oil.

2. A foodstuff according to claim 1, wherein said foodstuff has a firmness, suitably assessed as described in Test 1, of less than 10000 g, and has a firmness of at least 1000 g.

3. (canceled)

4. A foodstuff according to claim 1, wherein said oil is a vegetable oil and has a melting point of at least 20 °C and a melting point of less than 40 °C; and/or
wherein said oil includes at least 60 wt % of saturated fatty acids and less than 95 wt % of saturated fatty acids; and/or
said oil includes at least 4 wt % of monounsaturated fatty acids and less than 10 wt % of monounsaturated fatty acids; and/or
said oil includes less than 5 wt % of polyunsaturated fatty acids.

5. (canceled)

6. A foodstuff according to claim 1, wherein said oil includes:
at least 40 wt % of lauric acid and less than 65 wt % of lauric acid; and/or
at least 15 wt % of myristic acid and less than 30 wt % of myristic acid; and/or
at least 6 wt % of palmitic acid and less than 15 wt % of palmitic acid; and/or
at least 4 wt % of oleic acid and less than 10 wt % of oleic acid; and/or
at least 2 wt % of stearic acid and less than 5 wt % of stearic acid; and/or
at least 4 wt % of capric acid and less than 10 wt % of capric acid; and/or
at least 1 wt % of linoleic acid and less than 5 wt % of linoleic acid.

7. A foodstuff according to claim 1, wherein said oil is coconut oil.

8. A foodstuff according to claim 1, wherein:
said foodstuff includes at least 18 wt % of said oil and includes less than 40 wt % of said oil; and/or
the sum of the wt % of all oils and fats in said foodstuff excluding any oil/fat in the fungus is at least 21 wt % and said sum is less than 40 wt %;
wherein said fungus is a filamentous fungus, and optionally,
said fungus comprises filaments having number average lengths of less than 500 μm.

9-10. (canceled)

11. A foodstuff according to claim 8, wherein said fungus is a filamentous fungus which is a Fusarium species, optionally, Fusarium venenatum.

12. A foodstuff according to claim 11, wherein said foodstuff includes at least 1.0 wt % of said fungus on a dry mass basis; and optionally,
wherein said foodstuff includes at least 1.0 wt % of protein on a dry mass basis.

13. (canceled)

14. A foodstuff according to claim 1, wherein, in said foodstuff, the sum of the wt % of said fungus on a dry mass basis and said oil is at least 25 wt % and is less than 40 wt %; and/or
wherein, in said foodstuff, the sum of the wt % of each fungus on a dry mass basis and each oil/fat is at least 25 wt % and said sum is less than 40 wt %.

15. A foodstuff according to claim 8, wherein said foodstuff include at least 35 wt % of water and includes less than 60 wt % water.

16. A foodstuff according to claim 8, wherein, in said foodstuff:
a ratio defined as the weight of said oil divided by the weight of water is greater than 0.2 and the ratio is less than 3.0; and/or
a ratio defined as the total weight of oil(s)/fats(s) divided by the weight of water is greater than 0.2 and the ratio is less than 3.0.

17. A foodstuff according to claim 1, wherein said foodstuff includes:
2 to 15 wt %, of said fungus on a dry mass basis; and
20 to 40 wt % of said oil;
wherein said foodstuff includes one or more starches, wherein the sum of the wt % of starches in said foodstuff is at least 4.0 wt % and said sum is less than 20.0 wt %.

18. (canceled)

19. A foodstuff according to claim 17, wherein said foodstuff is a soft vegan cheese and includes at least 4.0 wt % or at least 7.0 wt % of a starch which has less than 10.0 wt % of amylose;
wherein said foodstuff includes a starch which includes at least 10 wt % of amylopectin and/or includes less than 10 wt %.

20. (canceled)

21. A foodstuff according to claim 17, wherein, in said foodstuff, the sum of the wt % of each fungus, each oil/fat, each starch and water is at least 70.0 wt % and said sum is less than 99.0 wt %.

22. A foodstuff according to claim 21, wherein said foodstuff includes less than 4.0 wt % of hydrocolloids in total.

23. A foodstuff according to claim 17, wherein said foodstuff includes:
less than 0.1 wt % of hydrocolloids in total; and/or
less than 4.0 wt % of non-starch based carbohydrate polymers in total; and/or
less than 4.0 wt % of sucrose; and/or
less than 4.0 wt %; and/or
less than 4.0 wt % of lactic acid producing bacteria;
wherein, in said foodstuff, the sum of the wt % of each fungus, each oil/fat, each starch, each flavourant, salt (eq NaCl) and water is at least 80.0 wt %.

24-25. (canceled)

26. A foodstuff according to claim 17, wherein said foodstuff is in the form of a block having a firmness, assessed as described in Test 1, in the range 1000 g to 10000 g.

27. A process for making a foodstuff according to claim 1, the process comprising mixing a fungus and an oil,
wherein said process is for producing a foodstuff having a firmness, assessed as described in Test 1, in the range 1000 g to 10000 g.

28. (canceled)

29. A foodstuff which is a vegan cheese, the foodstuff comprising:
a fungus; and
an oil;
wherein said oil is coconut oil;
wherein said fungus is a filamentous fungus;
wherein said foodstuff includes at least 1.0 wt % of said fungus on a dry mass basis;
wherein said foodstuff includes:
2 to 15 wt % of said fungus on a dry mass basis; and
20 to 40 wt % of said oil.

30. A foodstuff according to claim 29, wherein said foodstuff is in the form of a block having a firmness, assessed as described in Test 1, in the range 1000 g to 10000 g and said foodstuff includes:
3 to 6 wt % of said fungus on a dry mass basis; and
25 to 35 wt % of said oil;
wherein said foodstuff is a soft vegan cheese and includes at least 4.0 wt % of a starch which has less than 10.0 wt % of amylose;
wherein, in said foodstuff, the sum of the wt % of each fungus, each oil/fat, each starch and water is at least 70.0 wt % and said sum is less than 99.0 wt %;
wherein said foodstuff includes less than 4.0 wt % of hydrocolloids in total.
